(19) United States
(12) Patent Application Publication
(10) Pub. No.: US 2023/0137655 A1
(43) Pub. Date: May 4, 2023

(54) ANTIMICROBIAL COMPOSITIONS AND METHODS

(71) Applicant: CReDO Science, LLC, Austin, TX (US)

(72) Inventor: Ethan Russo, Vashon, WA (US)

(21) Appl. No.: 17/906,717

(22) PCT Filed: Mar. 19, 2021
(86) PCT No.: PCT/US21/23138

§ 371 Date: Sep. 19, 2022

Related U.S. Application Data
(60) Provisional application No. 62/992,800, filed on Mar. 20, 2020.

(51) Int. Cl. A01N 65/08; A01P 1/00

(52) U.S. Cl. CPC A01N 65/08; A01P 1/00

(57) ABSTRACT

The present invention relates to disinfecting products and compositions and formulations. More specifically, the invention relates to antimicrobial compositions, formulations containing the compositions, products containing the compositions or formulations, methods of preparation, and processes for disinfecting surfaces with the compositions or formulations.

### CLAIM OF PRIORITY UNDER 35 U.S.C. § 119

The present application for patent claims priority to Provisional Application No. 62/992,800 entitled “ANTIMICROBIAL COMPOSITIONS AND METHODS” filed Mar. 20, 2020 and assigned to the assignee hereof and hereby expressly incorporated by reference herein.

### BACKGROUND

### Field

The present invention relates to disinfecting products and compositions and formulations. More specifically, the invention relates to antimicrobial compositions, formulations containing the compositions, products containing the compositions or formulations, methods of preparation, and processes for disinfecting surfaces with the compositions or formulations.

### Background

A disinfectant refers to any chemical agent capable of killing, destroying, or inhibiting the growth of organisms, particularly microorganisms. Disinfectant products include antimicrobial compositions as hard surface cleaners, hand sanitizers, pre-disinfectant cleaners for instruments, sterilizing and high-level disinfectant compositions, and the like.

Ideally, a disinfectant has broad-spectrum activity against all types of microorganisms at various pH levels. The disinfectant should also have high efficacy so that a minimum amount of the anti-microbial agent can be used to save cost and to avoid or reduce any possible adverse effects caused by the anti-microbial agent. An ideal disinfectant is physically and chemically compatible with ingredients of different application systems so that the anti-microbial agent can suitably be incorporated in various products.

### SUMMARY

Some embodiments of the invention relate to an antimicrobial composition including a first compound derived from a Cannabis plant and a second compound derived from a Cannabis plant where the first compound derived from a Cannabis plant and the second compound derived from a Cannabis plant can exhibit synergistic antimicrobial effects. In some embodiments, the first compound derived from a Cannabis plant is a first cannabinoid and the second compound derived from a Cannabis plant is a second cannabinoid. In some embodiments, the first compound derived from a Cannabis plant is a cannabinoid and the second compound derived from a Cannabis plant is a terpene. In some embodiments, the first compound derived from a Cannabis plant is a first terpene and the second compound derived from a Cannabis plant is a second terpene.

Embodiments of the invention relate to an antimicrobial composition including a compound derived from a Cannabis plant capable of inhibiting or killing bacteria, yeast and/or fungi.

Embodiments of the invention relate to an antimicrobial composition including a compound derived from a Cannabis plant that can be employed in the field of cosmetic, toiletry, personal care, household, cleaning, disinfecting, food, beverages, enzyme formulations, feminine care products, foot care products, pet care products, food ingredients, paints, coatings, metal working fluids, nutrients for plants, construction and/or laundry products.

Some embodiments of the invention relate to a formulation including an antimicrobial composition including a compound derived from a Cannabis plant that is capable of being applied to a surface by spraying, rolling, fogging, or wiping.

Some embodiments of the invention relate to a formulation including an antimicrobial composition including a compound derived from a Cannabis plant in the form of a stick, roll-on, spray, pump-spray, aerosol, soap bar, powder, solution, gel, cream, wet wipes, balm or lotion.

Some embodiments of the invention relate to a method for preparing an antimicrobial composition that can include preparing a mixture of a carrier and at least one Cannabis derived component to yield a homogenous antimicrobial solution.

Some embodiments of the invention relate to a method for inhibiting or killing microbial growth on a surface including: mixing an effective amount of an antimicrobial composition including one Cannabis derived component with an appropriate product such as cosmetic, toiletry, personal care, household, cleaning, disinfecting, food, beverages, enzyme formulations, feminine care products, foot care products, pet care products, food ingredients, paints, coatings, metal working fluids, construction, nutrients for plants and/or laundry products, and applying the product to the surface.

### DETAILED DESCRIPTION

Antimicrobial compositions or formulations including the composition including at least one compound derived from a Cannabis plant are provided. The compound can be at least one of a cannabinoid or a terpene. The compound can be selected from pentyl, propyl, C-4, C-1 and monomethylether constituents of cannabinoid families, including but not limited to acidic and neutral forms of the cannabigerol (CBG), cannabichromene (CBC), cannabidiol (CBD), delta-9-tetrahydrohydrocannabinol (THC), delta-8-tetrahydrohydrocannabinol, cannabielsoin, cannabinol and cannabinodiol cannabinoid classes; and, cis and trans terpenoids or terpenes, including but not limited to myrcene, limonene, linalool, ocimene, beta-pinene, alphapinene, beta-caryophyllene, alpha-caryophyllene, delta-3-carene, ganmia-bisabolene, alpha-farnesene, beta-fenchol, guajol, alpha-guaiene, terpinolene, beta-eudesmol, alphabergamotene, epi-alpha-bisabolol and caryophyllene oxide; and/or the like.

“Compound derived from a Cannabis plant,” as used herein, can be defined as a compound naturally found in Cannabis. The actual compound used in the composition that is naturally found in Cannabis can be produced from another source.

In some embodiments, the composition can include CBD and/or CBG. In some embodiments, the composition can include beta-pinene and/or limonene.

In some embodiments, the composition or formulation including the composition can include about 1%, 5%, 10%, 15%, 20%, 25%, 30%, 35%, 40%, 45%, 50%, 55%, 60%, 65%, 70%, 75%, 80%, 85%, 90% or more cannabinoid.

In some embodiments, the composition or formulation including the composition can include about 0.001%, 0.01%, 0.1%, 1%, 5%, 10%, 15%, 20%, 25%, 30%, 35%, 40%, 45%, 50%, 55%, 60%, 65%, 70%, 75%, 80%, 85%, 90% or more terpenoid or terpene.

Where there are at least two components of the composition, or formulation including the composition, the two components together can have synergistic antimicrobial effects as provided in Table 1. For example, formula 1 includes a combination of CBG and CBD which exhibits synergistic antimicrobial effects. For example, formula 8 includes a combination of terpene and CBG which exhibits synergistic antimicrobial effects.

The use of the terms, “synergistic” and “synergistically effective,” are used in the present invention to mean a biological effect created from the application of two or more agents to produce a biological effect that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

TABLE 1
Synergistic combinations of Formulations

| | | | Non-CBD/CBG | | | |
|---|---|---|---|---|---|---|
| | CBD | CBG | Cannabinoid A | Terpene A | Terpene B | Carrier |
| CBD | | Formula 1 | Formula 2 | Formula 3 | Formula 4 | Formula 5 |
| CBG | Formula 6 | | Formula 7 | Formula 8 | Formula 9 | Formula 10 |
| Non-CBD/CBG | Formula 11 | Formula 12 | Formula 13 | Formula 14 | Formula 15 | Formula 16 |
| Cannabinoid 1 | | | | | | |
| Terpene 1 | Formula 17 | Formula 18 | Formula 19 | Formula 20 | Formula 21 | Formula 22 |
| Terpene 2 | Formula 23 | Formula 24 | Formula 25 | Formula 26 | Formula 27 | Formula 28 |
| Carrier | Formula 29 | Formula 30 | Formula 31 | Formula 32 | Formula 33 | |

Some embodiments relate to formulations comprising an emulsion which can be antimicrobial and can be used as, for example, surface cleansers, disinfectants, antiseptics, hand sanitizers, preservatives in animal feedstock and/or wound care preparations. In some embodiments, the formulation includes at least one compound derived from a Cannabis plant, a non-ionic or anionic surfactant, a polar solvent, and/or water. In some embodiments, the formulations can be manufactured using a self-assembly process, for example emulsions having a sub-micron mean emulsion particle size spontaneously or substantially spontaneously forming when ingredients are combined. The emulsions can be stable across a wide range of temperatures, and can have long storage life. In some embodiments, formulations can be effective antimicrobial agents against a host of pathogens, including pathogenic parasites, bacteria, fungi, viruses, viroids, virions, virusoids, prions, and/or endospores (collectively referred to herein as “microbes,” “microorganisms,” and/or “microbial pathogens”). The compositions can be non-staining and/or streak free, without leaving oily or soapy residues on surfaces to which the compositions are applied.

In some embodiments, antimicrobial compositions can be capable of reducing microorganism populations on contact with a variety of inanimate surfaces, living tissue and/or in foodstuffs. These microbes can include Gram-positive and/or Gram-negative bacteria as well as other microbial pathogens. The microorganisms can be loosely adhered to the surface or aggregated in a biofilm matrix. The compositions can be stable over an extended period of time. In some embodiments, the compositions can withstand boiling, freezing and and/or centrifugation, for example exhibiting nominal changes in emulsion droplet size. In some embodiments, shelf life of the composition can be at least about 3 months, 6 months, 1 year, or up to 3 years. In some embodiments, these compositions can be suitable for a variety of commercial applications.

The term “antimicrobial,” as used herein, describe the ability to inactivate and/or kill microbial pathogens. “Antiseptics” and “sanitizers” are defined as killing microbial pathogens within about 10 minutes or less, for example as specified by some U.S. government performance standards. Examples of bacterial microbial pathogens include gram positive bacteria and gram negative bacteria, for example, Mycobacterium tuberculosis, Salmonella enterica, Listeria monocytogenes, Escherichia coli, Clostridium botulinum, Clostridium difficile, Campylobacter, Bacillus cereus, Vibrio parahaemolyticus, Vibrio cholerae, Vibrio vulnificus, Staphylococcus aureus, Yersinia enterocolitica, shigella, and combinations thereof. Examples of virus types include enterovirus, norovirus, influenza, rotavirus, coronavirus and/or combinations thereof. Examples of parasites include Cryptosporidium, Toxoplasma gondii, Giardia duodenalis, Cyclospora cayetanensis, Trichinella spiralis, Taenia saginata, Taenia solium, and/or combinations thereof.

In some embodiments, the composition inactivates and/or kills greater than or equal to about 80, 85, 90, 95, 99, 99.9, 99.99, or 99.999% or more of microbial pathogens. In some embodiments, the composition inactivates and/or kills greater than or equal to about 80, 85, 90, 95, 99, 99.9, 99.99, or 99.999% or more of microbial pathogens in less than a period of time. The period of time can be 15, 10, 7, 5, or two minutes.

Some embodiments of the invention relate to a disinfectant formulation. In some embodiments, the disinfectant formulation is in a liquid form. The composition of the disinfectant formulation includes at least one compound derived from a Cannabis plant. The composition can further include a solvent (such as water or a low molecular weight alcohol), a surfactant, a colorant, a fragrance, and/or the like.

Some embodiments of the invention relate to a liquid composition that can be formulated to disinfect a surface. The formulation can be applied to a surface by spraying, rolling, fogging, wiping and/or the like. The formulation can be a surface disinfectant, killing infectious microbes present on the surface.

Various different disinfectant products can be made in accordance with the present disclosure. The disinfectant product may be used, for instance, to clean hard surfaces, to pre-clean sterilize or high-level disinfect instruments, and/or as a hand sanitizer. The liquid composition can include at least one compound derived from a Cannabis plant, a carrier such as a solvent, and other optional components such as fragrances.

The carrier or medium can be any solvent that is volatile and allows easy evaporation at ambient condition. Examples of liquid carriers include, but are not limited to, water and low molecular weight alcohols such as C1 to C8 alkanols. Specific examples include, but are not limited to, ethanol, isopropyl alcohol, butanol, pentanol, and/or the like and/or combinations thereof. In some embodiments, the liquid carrier can be ethanol.

Another class of solvents can include alkylene glycol ether. Examples include, but are not limited to, ethylene glycol monopropyl ether, ethylene glycol monobutyl ether, ethylene glycol monohexyl ether, ethylene clycol monohexyl ether, diethylene glycol monomethyl ether, diethylene glycol monoethyl ether, diethylene glycol monobutyl ether, diethylene glycol monohexyl ether, triethylene glycol monomethyl ether, triethylene glycol monoethyl ether, triethylene glycol monobutyl ether, propylene glycol methyl ether, propylene glycol methyl ether acetate, propylene glycol n-butyl ether, dipropylene glycol n-butyl ether, dipropylene glycol methyl ether, dipropylene glycol methyl ether acetate, propylene glycol n-propyl ether, dipropylene glycol n-propyl ether, tripropylene glycol methyl ether, and the like.

Another class of solvents include terpenes and their derivatives such as terpene alcohols, terpene esters, terpene ethers, or terpene aldehydes. Examples of solvents, include but are not limited to, pine oil, lemon oil, limonene, pinene, cymene, myrcene, fenchone, borneol, nopol, cineole, ionone, and/or the like, and/or combinations thereof.

In some embodiments, the compositions and formulations can consist of at least about 75%, 80%, 85%, 90%, 95%, 97%, or 100% organic material. “Organic” is defined as “of, relating to, or derived from living organisms.”

Some embodiments of the invention relate to application of the liquid formulation as a pressurized aerosol. In these embodiments, a propellant is included in the formulation. A variety of propellants or mixtures can be used for the present invention and should be familiar to those skilled in the art. C1 to C10 hydrocarbons or halogenated hydrocarbons are typical propellants in aerosol compositions known to the industry. Examples of such propellants include, but are not limited to, pentane, butane, propane, and methane. Other types of propellants that can be used include compressed air, nitrogen, or carbon dioxide. Alternatively, a Bag-on-Valve (BoV) package can be used to aerosol the product without directly adding a propellant to the composition.

Either a single solvent or a mixture of the above solvents can be used. Water can be mixed with the solvent. The types of solvents used for the present invention can depend upon the intended uses of the disinfectant composition. For example, if the composition is intended for home care use, cleaning the contaminated surfaces free of all types of dirt or soil can be of primary interest. Liquid carriers or media that assist and enhance the removal of soil can be the formulation. For example, the disinfectant formulation or composition can include alkyl or multi-alkyl glycol ethers for better cleaning performance in the home-care version of the formulation. On the other hand, if the primary goal of the disinfectant composition is to be used at a health care facility where the major concern is hospital-acquired infection, then quick drying of the liquid composition of the present invention may be more desirable than cleaning dirt or soil out of the surfaces. Low molecular weight alcohols should be considered to help the liquid formulation dry fast after the application. Also, a low molecular weight alcohol in the liquid formulation will strengthen the sanitizing activity of the liquid composition.

For health care use of the disinfectant, a mixture of water and low molecular weight alcohol can be used. The amount of alcohol present in the liquid formulation can be at such a level that the liquid formulation is capable of forming a zeotropic mixture between the alcohol and water. A minimum amount of alcohol, if present, in the liquid composition can be about 10%. For health care use, the alcohol concentration can be at least about 30%, or at least 50% based on the weight of liquid formulation for the health care use.

### Surfactant

In some embodiments, a surfactant or wetting agent can be used. The surfactant can assist the formulation to spread and evenly coat the surface being treated. The surfactant can additionally contribute to the formation of a zeotropic mixture between alcohol and water, thus facilitating a rapid and uniform drying of the liquid formulation once being applied onto surface.

Surfactants appropriate for the present liquid formulation can include, but are not limited to, those that are nonionic, anionic, or amphoteric in nature. Examples of commercially available wetting agents include, but are not limited to, Ecosurf SA-4 or Tergitol TMN-3 from Dow Chemical, and Q2-5211 from Dow Corning, and the like.

In some embodiments, the surfactant is organic. The surfactant can be any commercially available organic surfactants. For example, the surfactant can consist of Liquid Yucca Extract (Indian River Organics, Maitland, Fla.) or the like.

In the category of nonionic surfactants, ethoxylated alcohols with different amounts of ethylene oxides or HLB values can be used. Examples of ethoxylated alcohols include, but are not limited to, Triton X-100 (Dow Chemical, Midland Mich.), Ecosurf EH nonionic surfactant series from Dow Chemical, Tergitol nonionic surfactant series from Dow Chemical, the Surfonic surfactant series from Huntsman Corp., the Neodol surfactant series from Shell, the Ethox surfactant series from Ethox Chemicals, the Tomadol surfactant series from Air Products and Chemicals, Inc, and the like.

Another class of nonionic surfactants include alkylpolyglucosides. Examples include the Glucopon Series from BASF and the Ecoteric series from Huntsman and the like.

An alternative class of surfactants that is preferred for the liquid formulation are silane-based surfactants. Examples include but, are not limited to, silicone polyethers organofunctional or reactive silane wetting agents, fluorochemical based wetting agents, and the like.

The content of the surfactant in the liquid formulation is in a range of 0% to 10%. In some embodiments, the range can be 0.01% to 5%.

Depending on the targeted uses, a liquid formulation of the present invention for home care use may need appropriate pH condition. For example, if the liquid product is used in the kitchen area, a high pH product can be used in order to effectively remove grease soils commonly found in the area. If the product is used in bathroom area, soap scum and hard water deposits may be the primary concern. In such case, a low pH product can be used. There is no limitation on the types of pH adjusting agents that can be added into the liquid composition of the present invention. Example of pH adjusting agents that can be used include, but are not limited to, triethanolamine, diethanolamine, monoethanolamine, sodium hydroxide, sodium carbonate, potassium hydroxide, potassium carbonate, calcium carbonate, citric acid, acetic acid, hydrochloric acid, sulfamic acid, sulfuric acid and the like.

Other than components mentioned above, additional functional components can be included in the liquid composition of the present invention. Additional components can include, but are not limited to, chelants, compatibilizers, coupling agents, corrosion inhibitors, rheology modifiers, fragrances, colorants, preservatives, UV stabilizers, optical brighteners, active ingredient indicators, and the like.

In an embodiment of the present invention, the liquid solution includes a polymer binder, a quaternary ammonium compound, a silicone-based surfactant, and ethanol. The liquid formulation can be made or mixed by any conventional method known to one of ordinary skill in the art. There are no preferred addition procedures for the formulation of the present invention provided that the formulation is ultimately homogeneous, compatible and stable. For example, if the polymer binder is a solid, the polymer can be first dissolved or dispersed in a carrier such as water or alcohol to make a stock polymer binder liquid dispersion. The stock polymer binder liquid dispersion can be added into the formulation of the present invention during the mixing procedure.

### Application of Liquid Formulation

The liquid formulation can be applied by a variety of means. If sprayed, the liquid formulation can be used in a conventional bottle with a sprayer. The sprayer can be a trigger sprayer. As an alternative to a trigger sprayer, an aerosol can also be used to deliver the liquid formulation on to surfaces. Additional application modes include, but are not limited to, fogging, rolling, brushing, mopping, using a wipe by a variety of application devices, and/or the like. Wipe products can also be made including or pre-treated with the disinfectant composition/formulation(s), for example, for off-the-shelf sale or use.

Some embodiments of the invention relate to methods of using the composition to disinfect a contaminated surface. The method can include spraying the liquid formulation until the area is completely covered. The wet formulation subsequently can be wiped dry with a dry cloth or paper towel.

### EXAMPLES

### Example 1

Experiments are done to demonstrate the synergistic effects of the compounds in the composition.

Cells, such as RC-37 cells (African green monkey kidney cells), are grown in a monolayer culture as is routine in the art. The monolayers are removed and plated out onto culture plates for cytotoxicity and antiviral assays and propagated at 37 C in an atmosphere of 5% C02.

Viruses, such as coronavirus, are grown on the cells and virus stock cultures are prepared from supernatants of infected cells and stored at −80 C. Infectivity titers are determined by a standard plaque assay on confluent cells.

Example methods can be found in Appendino G, Gibbons S, Giana A, et al. Antibacterial cannabinoids from Cannabis sativa: a structure-activity study. J Nat Prod. 2008; 71(8):1427-1430. doi:10.1021/np8002673; Astani A, Schnitzler P. Antiviral activity of monoterpenes beta-pinene and limonene against herpes simplex virus in vitro. Iran J Microbiol. 2014; 6(3):149-155; Astani A, Reichling J, Schnitzler P. Screening for antiviral activities of isolated compounds from essential oils. Evid Based Complement Alternat Med. 2011; 2011:253643; Blaskovich M A T, Kavanagh A M, Elliott A G, et al. The antimicrobial potential of cannabidiol. Commun Biol. Jan. 19 2021; 4(1):7; Salehi B, Upadhyay S, Erdogan Orhan I, et al. Therapeutic Potential of alpha- and beta-Pinene: A Miracle Gift of Nature. Biomolecules. Nov. 14 2019; 9(11); and Farha M A, El-Halfawy O M, Gale R T, et al. Uncovering the Hidden Antibiotic Potential of Cannabis. ACS Infect Dis. Mar. 13 2020; 6(3):338-346; the entire contents of the foregoing references are incorporated by reference in their entirety.

The tested compositions include (1) the compositions of the invention with both the two synergistic compounds, (2) the composition with only the first compound, (3) the composition with the with only the second compound, and (4) a control composition with neither of the compounds. The control composition is otherwise identical to the other compositions, such as in terms of pH, solvents, carries, surfactants, etc.

### Example 2

Cytotoxicity assays are performed to test the compositions of Example 1. Each composition is added onto cells in replicates for each formulation. After 3 days of incubation, the growth medium is removed and viability of the formulation-treated cells is determined in a standard cytotoxicity assay.

### Example 3

Dose-response response assays are performed to test the compositions of Example 1. Inhibition of viral replication is measured by plaque reduction assays. Plaque forming units (pfu) are incubated with different formulations and then virus is allowed to adsorb to the cells. The formulation is discarded and infected cells are overlaid with medium. Each formula is performed in replicate. Virus-infected cells in wells containing medium with 1% ethanol but no formula are also included on each plate as controls. After incubation, monolayers are fixed. The cultures are stained and plaques are counted. The concentrations of test compound which inhibit plaque numbers by 50% (IC50) is determined from dose-response curves.

### Example 4

Time of addition assay are performed using the compositions of Example 1. Cells are pretreated with composition before viral infection, viruses are incubated with drugs before infection or infected cells are incubated with composition immediately after penetration of the virus into cells.

### Example 5

Addition of compositions of Example 1 during viral intracellular replication is also tested. The effect of the composition is tested during the replication period by addition of compositions after cell infection to the overlay medium, as typically performed in antiviral susceptibility studies. Each assay is run in replicates. Plaque reduction assays are carried out as described above and number of plaques of drug-treated cells and viruses are compared to untreated controls. Viruses are synchronized at each stage of infection. The composition-pretreated cell free viruses are added at the same time to host cells, thus infection starts at the same time. When intracellular viruses are drug treated, the infection starts simultaneously, all non-infecting virus particles having been washed off and all intracellular viruses being in the same phase of replication.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. An antimicrobial composition comprising a first compound derived from a Cannabis plant and a second compound derived from a Cannabis plant, wherein the first compound derived from a Cannabis plant and the second compound derived from a Cannabis plant exhibit synergistic antimicrobial effects.

2. The antimicrobial compound of claim 1, wherein the first compound derived from a Cannabis plant is a first cannabinoid and the second compound derived from a Cannabis plant is a second cannabinoid.

3. The antimicrobial compound of claim 1, wherein the first compound derived from a Cannabis plant is a cannabinoid and the second compound derived from a Cannabis plant is a terpene.

4. The antimicrobial compound of claim 1, wherein the first compound derived from a Cannabis plant is a first terpene and the second compound derived from a Cannabis plant is a second terpene.

5. The antimicrobial composition of claim 1, wherein the composition is capable of inhibiting or killing bacteria, yeast and/or fungi.

6. The antimicrobial composition according to claim 1 employed in the field of cosmetic, toiletry, personal care, household, cleaning, disinfecting, food, beverages, enzyme formulations, feminine care products, foot care products, pet care products, food ingredients, paints, coatings, metal working fluids, nutrients for plants, construction and/or laundry products.

7. A formulation comprising the composition claim 1, wherein the formulation is capable of being applied to a surface by spraying, rolling, fogging, or wiping.

8. The formulation of claim 7 in the form of a stick, roll-on, spray, pump-spray, aerosol, soap bar, powder, solution, gel, cream, wet wipes, balm or lotion.

9. (canceled)

10. A method for inhibiting or killing microbial growth on a surface comprising
mixing an effective amount of antimicrobial composition of claim 1 with an appropriate product selected from the group consisting of cosmetic, toiletry, personal care, household, cleaning, disinfecting, food, beverages, enzyme formulations, feminine care products, foot care products, pet care products, food ingredients, paints, coatings, metal working fluids, construction, nutrients for plants and/or laundry products, and
applying the product to the surface.
